(19) United States
(12) Patent Application Publication
(10) Pub. No.: US 2004/0136941 A1
(43) Pub. Date: Jul. 15, 2004

(54) Cosmetic and dermatological preparation for removing sebum or regulating sebum production

(75) Inventors: Heiner Max, Hamburg (DE); Jens Nielsen, Henstedt-Ulzburg (DE); Thomas Raschke, Pinneberg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/715,603

(22) Filed: Nov. 17, 2003

Related U.S. Application Data
(63) Continuation of application No. PCT/EP02/05375, filed on May 16, 2002.

(30) Foreign Application Priority Data
May 17, 2001 (DE) 101 23 989.0

(51) Int. Cl. A61K 7/06; A61K 7/11

(52) U.S. Cl. 424/70.13

(57) ABSTRACT

The invention is a cosmetic or dermatological preparation for removing or regulating sebum that includes salts of hard trivalent or tetravalent metals combined with oligo- or polysaccharides. The present invention is also a method of applying the cosmetic or dermatological preparation to the skin or hair for removing sebum or regulating sebum production.

### CROSS-REFERENCE TO RELATED APPLICATIONS

This is a continuation application of PCT/EP02/05375, filed May 16, 2002, which is incorporated herein by reference in its entirety, and also claims the benefit of German Priority Application No. 101 23 989.0, filed May 17, 2001.

### FIELD OF THE INVENTION

The present invention relates to the use of topical preparations for removing sebum from the skin, and in particular for removing and clearing comedones, for avoiding comedone formation, for the prophylaxis and treatment of mild forms of acne, and for the prophylaxis and control of seborrhea.

### BACKGROUND OF THE INVENTION

Sebaceous gland overproduction and the possible skin disorders which develop as a result, such as sebum retention, formation of comedones in the region of the sebaceous glands, i.e., on the face (primarily forehead, nose and chin) and on the upper back, and consequently the various forms of acne, are a commonly encountered skin problem which has hitherto not been solved to a satisfactory degree.

Sebum is the secretion of the sebaceous gland. Sebaceous glands are protrusions of the follicle epithelium and thus constituents of the follicle with which they form a functional unit. They are holocrine glands, i.e., the entire gland cell is for the greatest part converted into the secretion sebum. The cells of the sebaceous gland, which are continually renewed from below, become fat and decompose, and the skin sebum formed as a result is emptied onto the surface of the skin through the follicle openings.

According to G. Leonardy (J.Ft. Jellinek Kosmetologie, Zweck und Aufbau kosmetischer Prätparate [Purpose and structure of cosmetic preparations], Dr. Alfred Hüthig-Verlag, Heidelberg-Mainz-Basel, third completely revised and significantly expanded edition 1976, pages 26 to 29), skin sebum consists of mono-, di- and triglycerides (C₁₀-C₁₈), waxes (C₁₆-C₂₆), wax esters (C₂₈-C₃₈), normal saturated fatty acids (C₁₀-C₁₈), branched-chain saturated fatty acids (C₁₀-C₁₈), multibranched-chain saturated fatty acids (C₁₃-C₁₈), monounsaturated fatty acids (C₁₀-C₁₈), polyunsaturated fatty acids (C₁₃, C₁₅-C₁₇), sterols (cholesterol, 7-dehydrocholesterol, 7-hydroxycholesterol), branched and unbranched hydrocarbons (C₃₀-C₄₀), squalene and phospholipids.

Together with the aqueous secretion of the eccrine sweat glands, the lipids of the sebaceous glands form the hydro-lipid film of the skin. This surface film is an emulsion which may be a water-in-oil or an oil-in-water emulsion. It has the function of keeping the surface of the skin supple and of regulating the water content of the deeper layers of the skin. If the sebum is well hydrated, the water content is at least 10 to 20% by weight and the sebum is hydrophilic. If the hydrophilic-lipophilic balance of the surface film is disturbed and the water content decreases, the sebum changes and becomes hydrophobic. The flow of sebum from the sebaceous glands and follicles is impeded. This results in the skin sebum being trapped in the follicle openings with this then, as a result, possibly leading to comedones and inflammation of the follicles.

The change in the skin sebum and the onset of comedone formation can have various causes. For example: external influences, such as incorrect cleansing habits and incorrect care, comodogenous substances in cosmetics, weathering influences, alkaline soaps and harsh detergents. Increased sebaceous gland secretion and the formation of comedones can also develop as a result of genetic factors and hormonal influences. Here too, comedones, inflammations, pre-acne and acne with its secondary effects may be the result.

The frequency of skin damage as a result of disturbed sebaceous gland function and disorders of the sebaceous glands is ever increasing, and the reversal/avoidance of comedone formation is thus a pressing concern. However, attempts hitherto to solve comedone formation as a causal problem have led to results which are less than satisfactory.

As well as the manual removal of comedones by squeezing, numerous cleansing methods are known with which it is attempted to remove comedones and to permanently prevent comedone formation. These include special soaps, skin-peeling compositions and the like. Softening and astringent compositions are also used. Moreover, it is attempted to reduce the tendency for acne by adding drying, keratolytic, antiseborrheic and antibacterial active ingredients to cosmetic and pharmaceutical preparations, without irritations of the skin or drying of the skin arising.

However, skin cleansing degreases the skin and extracts moisture therefrom. In addition, soaps have the disadvantage that the water-insoluble calcium and magnesium salts of higher fatty acids, which form when the soaps are used in hard water, form slimy precipitates on the skin. Because they are difficult to rinse off, these precipitates remain for a relatively long period on the skin, block the follicle openings and can lead to the formation of comedones. For this reason, syndets (i.e. surfactants without soap character) are predominantly used in the form of washing creams or washing lotions for skin cleansing. Although these syndets do not form lime soaps, the treatment with highly surface-active agents has a greater degreasing and drying action than soap on the skin. The more often soap- and surfactant-containing products are applied to the skin, the more markedly their disadvantageous effects come to the fore, namely degreasing and drying of the skin as a result of destruction of the hydro-lipid film. The reduction in comedones almost always leads to a reduction in the water content in the upper layers of the skin and to a solid concrement formation in the sebaceous glands, which in turn can induce inflammation. However, the reduction in the content of moisture of the skin is counterproductive for a gentle removal of the comedones.

Seborrhea is an increased function of the sebaceous glands as a result of predisposition. Both scalp and skin on the face appear greasy. The composition of the seborrheic sebum is changed as compared with normal sebum. Three development stages of seborrhea are distinguished:

1. Simple seborrhea, mild cases, greasy after 8 days.

2. Oily seborrhea, greasy after just 2-3 days.

3. Irreversible form, can no longer be reversed. The seborrhea in which the hair appears to be bathed in grease after just one day.

The excessive secretion of the sebaceous glands can, inter alia, be triggered by androgenetic disorders and has a detrimental esthetic effect on the overall appearance of the hair. This disorder can also be the cause for alopecia which arises. A forerunner is in each case the seborrheic condition of the scalp. Vegetative disorders and inappropriate care can further worsen the appearance of the skin and also the condition of the hair. Even in cases of seborrhea, the hair may itself be dry as a result of disturbances in keratin formation. Dry, damaged hair is frequently caused by external stress such as, for example, sun or chemical treatments. Blow drying at too high a temperature or incorrect care of affected hair can lead to damage.

The causes of greasy hair lie within the human body and are hormonally determined. Each hair has its own sebaceous gland which produces grease (sebum). Sebum production is controlled hormonally, and over- or under-production may result, depending on the hormone sensitivity of the sebaceous gland. The sebum itself has the function of keeping the scalp supple. It passes from the sebaceous gland onto the scalp and only later to the hair root, where it is normally taken up by the hair shaft and remains invisible. In the case of the overproduction of sebum, the hair shaft is no longer able to accommodate this and the sebum becomes visible as a greasy film on the hair. The result is straggly, greasy-lustrous hair.

As a result of the fact that sebaceous gland production is dependent on the hormone balance, the problem of greasy hair cannot be solved fundamentally since the sebaceous glands continually produce grease. Consistent care and high-quality grooming routines continue to be the best method of controlling greasy hair.

Greasy hair has very troublesome consequences. The hair becomes straggly again just a short time after washing and the hairstyle does not hold.

Contrary to popular opinion, it is only a rumor that the hair becomes greasy more quickly as a result of excessively frequent washing. Mild shampoos for greasy hair ensure that excess grease is removed. Hair and scalp are supplied with sufficient moisture and counterbalance the overproduction of the sebaceous glands.

Greasy hair and dandruff are among the most common hair problems. These anomalies are to be attributed to a disturbance of sebaceous gland activity. If the sebaceous glands are hyperactive, the term used is seborrhea. In this connection, two forms can be differentiated: the oily form (seborrhea oleosa) and the dry form (seborrhea sicca).

Seborrhea Oleosa:

Here, the sebaceous glands are hyperactive, producing too much, and excessively oily, sebum. The skin therefore has a greasy shine, and the hair is greasy and straggly again just 2 to 3 days after washing, right into the ends.

Seborrhea Sicca:

This is likewise to be attributed to sebaceous glands being hyperactive, but the sebum is drier, and has a more solid consistency. With the small flakes of the epidermis, it forms large sebum flakes which are readily friable. The scalp has a wax-like shine, the hair becomes greasy only at the roots, and the lengths and particularly the ends are dry and even brittle.

The treatment of seborrhea involves, firstly, regular and thorough washing of the head with special shampoos, which can be carried out as often as appears necessary. The washing should be combined with a massage in the connective tissue because this empties the sebaceous glands much more, which delays regreasing.

### SUMMARY OF THE INVENTION

The object of the present invention is to provide a preparation which does not have the disadvantages of the known compositions used hitherto, which dissolves sebum and skin grease in a targeted manner and thus prevents the formation of comedones and the development of acne, at the same time removes existing comedones, and improves existing acne and additionally reduces the production of sebum and skin grease by the sebaceous glands.

This object is achieved according to the invention through the simultaneous use of salts of hard trivalent or tetravalent metal anions and unbranched, branched, cyclic or crosslinked oligosaccharides or polysaccharides, and derivatives thereof for producing preparations for the removal of and reduction in the production of sebum and grease on the skin.

Surprisingly, use of the preparation cancels the comedogenic effect of the raw materials used in the preparation, and thus the formation of comedones and, accordingly, the development of acne, is prevented.

It has also been found that the active ingredients used according to the invention simultaneously reduce the production of sebum and grease on the skin and prevent or, where applicable, eliminate the formation of seborrheic phenomena, in particular greasy hair, but also dandruff.

### DETAILED DESCRIPTION OF THE PREFERRED EMBODIMENTS

Suitable salts of hard trivalent or tetravalent metal ions to be used according to the invention are, for example, aluminum chlorohydrates. These are colorless, hygroscopic crystals which readily deliquesce in the air and are produced during the evaporation of aqueous aluminum chloride solutions. Aluminum chlorohydrate is used for producing antiperspirant and deodorizing preparations and is probably effective by partially closing the sweat glands by protein precipitation and/or polysaccharide precipitation. As well as the chlorohydrates, it is also possible to use aluminum hydroxylactates and acidic aluminum/zirconium salts.

Unbranched, branched, cyclic or crosslinked oligosaccharides or polysaccharides and derivatives thereof according to the invention are, for example, distarch phosphate or cyclodextrins (cycloamyloses, cycloglucans). These are known per se in cosmetic and pharmaceutical preparations. These substances are often used for “molecular encapsulation”, i.e. as a protective coating of sensitive molecules. Distarch phosphate is prepared by crosslinking starch with sodium metaphosphate. On the other hand, cyclodextrins are made up of 6, 7, 8 or even more α-1,4-linked glucose units, cyclohexaamylose (α-cyclodextrin) being characterized by the structure

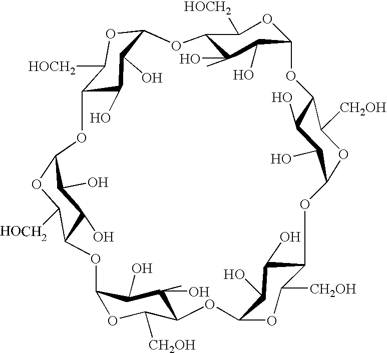

Cycloheptaamylose (β-cyclodextrin) is characterized by the structure

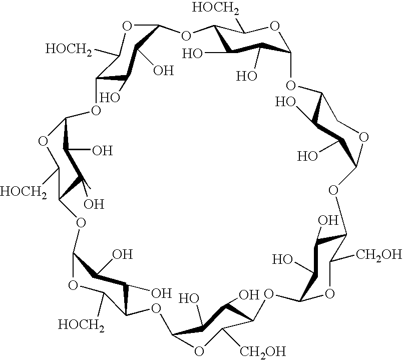

Cyclooctaamylose (γ-cyclodextrin) is characterized by the structure

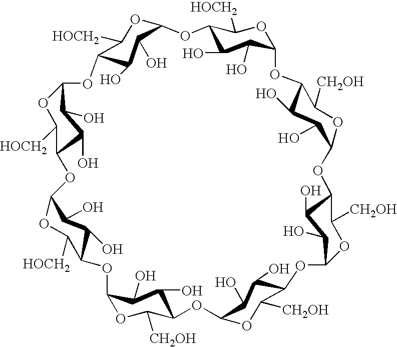

Cycloenneaamylose (δ-cyclodextrin) is characterized by the structure

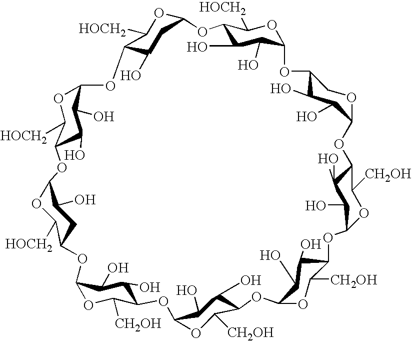

Within the scope of this patent, polar- and nonpolar-substituted cyclodextrins can also be used. These preferably include, but not exclusively, methyl-, ethyl- and hydroxypropyl-cyclodextrin.

The invention thus also provides a method for controlling blemished skin, acne, or seborrheic phenomena, in particular greasy hair and/or dandruff, characterized in that the active ingredients used according to the invention are brought, in a suitable cosmetic or dermatological carrier, into contact with the area affected by increased sebum production.

Further preferred embodiments of the present invention are thus formulations to be used against dandruff, for example antidandruff shampoos.

The prior art did not give the slightest indication of the use according to the invention of the active ingredients according to the invention as antiseborrheic or sebum-regulating active principle.

It is advantageous according to the invention if the cosmetic or dermatological preparations comprise 0.1-10% by weight of unbranched, branched, cyclic or crosslinked oligosaccharides or polysaccharides or derivatives thereof, and the total amount of the hard salts of trivalent or tetravalent metals used according to the invention in the finished cosmetic or dermatological preparations is chosen advantageously from the range 0.01-10% by weight, preferably 0.05-7% by weight, in particular 0.1-5% by weight, based on the total weight of the preparations.

The combination according to the invention of unbranched, branched, cyclic or crosslinked oligosaccharides or polysaccharides or derivatives thereof with hard salts of trivalent or tetravalent metals is, for the purposes of this specification, also referred to collectively as “active ingredient according to the invention” or “active ingredient used according to the invention” or “active ingredient combination used according to the invention” or are given synonymous designations.

The active ingredient combinations according to the invention or cosmetic or dermatological preparations comprising such active ingredient combinations are entirely satisfactory preparations in every respect. It could not have been foreseen by the person skilled in the art that the preparations according to the invention

better reduce the production of sebum,

better remove sebum from the surface of the skin,

better prevent the formation of acne,

are better suitable for the treatment of damaged skin,

better counteract seborrheic skin conditions and

better matt the skin than the preparations of the prior art.

According to the invention, customary antioxidants may be added to preparations which comprise the active ingredient combinations according to the invention.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoides, carotenes (e.g. α-carotene, β-carotene, lycopene) and derivatives thereof, ubiquinones and derivatives thereof, aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin) α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, alaninediacetic acid, flavonoids, polyphenols, catechins, vitamin C and derivatives (e.g. ascorbyl palmitate, Mg ascorbyl phosphate, ascorbyl acetate) tocopherols and derivatives (e.g. vitamin E acetate), and coniferyl benzoate of benzoin resin, rutinic acid and derivatives thereof, ferulic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of these said active ingredients which are suitable according to the invention.

The amount of antioxidants (one or more compounds) in the preparations is preferably 0.001 to 30% by weight, particularly preferably 0.05-20% by weight, in particular 1-10% by weight, based on the total weight of the preparation.

The prophylaxis or the cosmetic or dermatological treatment with the active ingredient used according to the invention or with the cosmetic or topical dermatological preparations with an active content of active ingredient used according to the invention is carried out in the usual manner, by applying the active ingredient used according to the invention or the cosmetic or topical dermatological preparations with an active content of active ingredient used according to the invention to the affected areas of skin.

The active ingredient used according to the invention can advantageously be incorporated into customary cosmetic and dermatological preparations, which may be in various forms. Thus, they may, for example, be a solution, an emulsion of the water-in-oil (W/O) type or of the oil-in-water (O/W) type, or multiple emulsions, for example of the water-in-oil-in-water (W/O/W) type or oil-in-water-in-oil (O/W/O) type, a hydrodispersion or lipodispersion, a gel, a solid stick or an aerosol.

Emulsions according to the invention for the purposes of the present invention, e.g., in the form of a cream, a lotion, a cosmetic milk, are advantageous and comprise, for example, fats, oils, waxes and/or other fatty substances, and water and one or more emulsifiers as are customarily used for this type of formulation.

It is also possible and advantageous for the purposes of the present invention to incorporate the active ingredient used according to the invention into aqueous systems or surfactant preparations for cleansing the skin and the hair.

The person skilled in the art is of course aware that demanding cosmetic compositions are mostly inconceivable without the customary auxiliaries and additives. The cosmetic preparations according to the invention can therefore comprise cosmetic auxiliaries, as are customarily used in such preparations, e.g. preservatives, bactericides, deodorizing substances, antiperspirants, insect repellents, vitamins, antifoams, dyes, pigments with a coloring action, thickeners, softening substances, moisturizing substances and/or humectant substances, fats, oils, waxes or other customary constituents of a cosmetic formulation, such as alcohols, polyols, polymers, foam stabilizers, electrolytes, organic solvents or silicone derivatives.

Corresponding requirements apply mutatis mutandis to the formulation of medicinal preparations.

Medicinal topical compositions for the purposes of the present invention generally comprise one or more medicaments in an effective concentration. For the sake of simplicity, for a clear distinction between cosmetic and medicinal application and corresponding products, reference is made to the legal provisions of the Federal Republic of Germany (e.g. Cosmetics Directive, Foods and Drugs Act).

Preparations according to the invention can advantageously further comprise substances which absorb UV radiation in the UVB region, where the total amount of filter substances is, for example, 0.1% by weight to 30% by weight, preferably 0.5 to 10% by weight, in particular 1.0 to 6.0% by weight, based on the total weight of the preparations, in order to provide cosmetic preparations which protect the hair and/or the skin from the entire range of ultraviolet radiation. They can also serve as sunscreens for the hair.

If the preparations according to the invention comprise UVB filter substances, these may be oil-soluble or water-soluble. Oil-soluble UVB filters which are advantageous according to the invention are, for example:

3-benzylidenecamphor derivatives, preferably 3-(4-methylbenzylidene)camphor, 3-benzylidenecamphor;

4-aminobenzoic acid derivatives, preferably 2-ethylhexyl4-(dimethylamino)benzoate, amyl 4-(dimethylamino)benzoate;

esters of cinnamic acid, preferably 2-ethylhexyl4-methoxycinnamate, isopentyl 4-methoxycinnamate;

esters of salicylic acid, preferably 2-ethylhexyl salicylate, 4-isopropylbenzyl salicylate, homomenthyl salicylate,

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably di(2-ethylhexyl) 4-methoxybenzal-malonate, and

2,4,6-trianilino(p-carbo-2′-ethyl-1′-hexyloxy)-1,3,5-triazine.

Advantageous water-soluble UVB filters are, for example:

salts of 2-phenylbenzimidazole-5-sulfonic acid, such as its sodium, potassium or its triethanolammonium salt, and the sulfonic acid itself;

sulfonic acid derivatives of benzophenones, preferably 2-hydroxy-4-methoxybenzophenone-5-sulfonic acid and its salts; and

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and its salts, and 1,4-di(2-oxo-10-sulfo-3-bornylidenemethyl)benzene and its salts (the corresponding 10-sulfato compounds, for example the corresponding sodium, potassium or triethanolammonium salt), also referred to as benzene-1,4-di(2-oxo-3-bornylidenemethyl-10-sulfonic acid.

The list of said UVB filters which can be used in combination with the active ingredient combinations according to the invention is of course not intended to be limiting.

It may also be advantageous to use UVA filters which are customarily present in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. It is possible to use the amounts used for the UVB combination.

Cosmetic and dermatological preparations according to the invention advantageously also comprise inorganic pigments based on metal oxides and/or other metal compounds which are insoluble or sparingly soluble in water, in particular the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. The pigments are particularly preferably based on TiO₂.

For the purposes of the present invention, it is particularly advantageous, although not obligatory, for the inorganic pigments to be present in hydrophobic form, i.e. to have been treated on the surface to repel water. This surface-treatment may involve providing the pigments with a thin hydrophobic layer by processes known per se.

One such process involves, for example, producing the hydrophobic surface layer in accordance with a reaction according to

n TiO₂+m(RO)₃Si—R′—>n TiO₂ (surf.)

Here, n and m are stoichiometric parameters to be used as desired, R and R′ are the desired organic radicals. For example, hydrophobicized pigments prepared analogously to DE-A 33 14 742 are advantageous.

Advantageous TiO₂ pigments are available, for example, under the trade names MT 100 T from TAYCA, and also M 160 from Kemira and T 805 from Degussa.

Preparations according to the invention may, especially when crystalline or microcrystalline solid bodies, for example inorganic micropigments, are to be incorporated into the preparations according to the invention, also comprise anionic, non-ionic and/or amphoteric surfactants. Surfactants are amphiphilic substances which can dissolve organic, nonpolar substances in water.

The hydrophilic moieties of a surfactant molecule are mostly polar functional groups, for example —COO⁻, —OSO₃²⁻, —SO₃⁻, whereas the hydrophobic moieties are usually nonpolar hydrocarbon radicals. Surfactants are generally classified according to the type and charge of the hydrophilic molecular moiety. In this connection, it is possible to differentiate between four groups:

anionic surfactants,

cationic surfactants,

amphoteric surfactants and

nonionic surfactants.

Anionic surfactants usually have, as functional groups, carboxylate, sulfate or sulfonate groups. In aqueous solution, they form negatively charged organic ions in acidic or neutral medium. Cationic surfactants are characterized almost exclusively by the presence of a quaternary ammonium group. In aqueous solution, they form positively charged organic ions in acidic or neutral medium. Amphoteric surfactants contain both anionic and cationic groups and accordingly in aqueous solution exhibit the behavior of anionic or cationic surfactants depending on the pH. In strongly acidic medium, they have a positive charge, and in alkali medium a negative charge. By contrast, in the neutral pH range, they are zwitterionic, as the example below is intended to illustrate:

RNH₂⁺CH₂CH₂COOH X⁻ (at pH=2) X⁻=any anion, e.g. Cl⁻

RNH₂⁺CH₂CH₂COO⁻ (at pH=7)

RNHCH₂CH₂COO⁻ B⁺ (at pH=12) B⁺=any cation, e.g. Na⁺

Typical non-ionic surfactants are polyether chains. Non-ionic surfactants do not form ions in aqueous medium.

A. Anionic Surfactants.

Anionic surfactants which can be used advantageously are acylamino acids (and salts thereof), such as

1. acyl glutamates, for example sodium acyl glutamate, di-TEA-palmitoyl aspartate and sodium caprylic/capric glutamate,

2. acylpeptides, for example palmitoyl-hydrolyzed milk protein, sodium cocoyl-hydrolyzed soya protein and sodium/potassium cocoyl-hydrolyzed collagen,

3. sarcosinates, for example myristoyl sarcosine, TEA-lauroyl sarcosinate, sodium lauroyl sarcosinate and sodium cocoyl sarcosinate,

4. taurates, for example sodium lauroyl taurate and sodium methyl cocoyl taurate,

5. acyl lactylates, lauroyl lactylate, caproyl lactylate

6. alaninates carboxylic acids and derivatives, such as

a. carboxylic acids, for example lauric acid, aluminum stearate, magnesium alkanolate and zinc undecylenate,

b. ester carboxylic acids, for example calcium stearoyl lactylate, laureth-6 citrate and sodium PEG-4 lauramide carboxylate,

c. ether carboxylic acids, for example sodium laureth-13 carboxylate and sodium PEG-6 cocamide carboxylate,

phosphoric esters and salts, such as, for example, DEA-oleth-10 phosphate and dilaureth-4 phosphate,

sulfonic acids and salts, such as

1. acyl isethionates, e.g. sodium/ammonium cocoyl isethionate,

2. alkylarylsulfonates,

3. alkylsulfonates, for example sodium cocomonoglyceride sulfate, sodium C₁₂₋₁₄-olefinsulfonate, sodium lauryl sulfoacetate and magnesium PEG-3 cocamide sulfate,

4. sulfosuccinates, for example dioctyl sodium sulfosuccinate, disodium laureth sulfosuccinate, disodium lauryl sulfosuccinate and disodium undecyleneamido-MEA sulfosuccinate and

sulfuric esters, such as

1. alkyl ether sulfates, for example sodium, ammonium, magnesium, MIPA, TIPA laureth sulfate, sodium myreth sulfate and sodium C₁₂₋₁₃ parethsulfate,

2. alkyl sulfates, for example sodium, ammonium and TEA lauryl sulfate.

B. Cationic Surfactants

Cationic surfactants which can be used advantageously are

1. alkylamines,

2. alkylimidazoles,

3. ethoxylated amines and

4. quaternary surfactants

5. ester quats

Quaternary surfactants comprise at least one N atom which is covalently bonded to 4 alkyl and/or aryl groups. Irrespective of the pH, this leads to a positive charge. Alkylbetaine, alkylamidopropylbetaine and alkylamidopropylhydroxysulfain are advantageous quaternary surfactants. The cationic surfactants used according to the invention can also be preferably chosen from the group of quaternary ammonium compounds, in particular benzyltrialkylammonium chlorides or bromides, such as, for example, benzyldimethylstearylammonium chloride, and also alkyltrialkylammonium salts, for example cetyltrimethylammonium chloride or bromide, alkyldimethylhydroxy-ethylammonium chlorides or bromides, dialkyldimethylammonium chlorides or bromides, alkylamidoethyltrimethylammonium ether sulfates, alkylpyridinium salts, for example lauryl- or cetylpyrimidinium chloride, imidazoline derivatives and compounds with a cationic character, such as amine oxides, for example alkyl dimethylamine oxides or alkylaminoethyldimethylamine oxides. In particular, the use of cetyltrimethylammonium salts is advantageous.

C. Amphoteric Surfactants

Amphoteric surfactants which can be used advantageously are

1. acyl/dialkylethylenediamine, for example sodium acyl amphoacetate, disodium acyl amphodipropionate, disodium alkyl amphodiacetate, sodium acyl amphohydroxypropylsulfonate, disodium acyl amphodiacetate and sodium acyl amphopropionate,

2. N-alkylamino acids, for example aminopropylalkylglutamide, alkylaminopropionic acid, sodium alkylimidodipropionate and lauroamphocarboxyglycinate.

D. Nonionic Surfactants

Nonionic surfactants which can be used advantageously are

1. alcohols,

2. alkanolamides, such as cocamides MEA/DEA/MIPA,

3. amine oxides, such as cocoamidopropylamine oxide,

4. esters which are formed by esterification of carboxylic acids with ethylene oxide, glycerol, sorbitan or other alcohols,

5. ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and cocoglycoside

6. sucrose esters, sucrose ethers

7. polyglycerol esters, diglycerol esters, monoglycerol esters

8. methyl glucose esters, esters of hydroxy acids

Also advantageous is the use of a combination of anionic and/or amphoteric surfactants with one or more nonionic surfactants.

The surface-active substance may be present in the preparations according to the invention in a concentration between 1 and 95% by weight, based on the total weight of the preparations.

The lipid phase of the cosmetic or dermatological emulsions according to the invention can advantageously be chosen from the following group of substances:

mineral oils, mineral waxes

oils, such as triglycerides of capric or of caprylic acid, and also natural oils such as, for example, castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alkyl benzoates; and

silicone oils, such as dimethylpolysiloxanes, diethylpolysiloxanes, diphenylpolysiloxanes and mixed forms thereof.

The oil phase of the emulsions of the present invention is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, of silicone oils, of dialkyl ethers, the group of saturated or unsaturated, branched or unbranched alcohols, and the fatty acid triglycerides, namely the triglycerol esters of saturated and/or unsaturated, branched and/or unbranched alkanecarboxylic acids having a chain length of from 8 to 24, in particular 12-18, carbon atoms. The fatty acid triglycerides can, for example, advantageously be chosen from the group of synthetic, semisynthetic and natural oils, e.g. olive oil, sunflower oil, soybean oil, groundnut oil, rapeseed oil, almond oil, palm oil, coconut oil, palm kernel oil and the like.

Any mixtures of such oil and wax components can also be used advantageously for the purposes of the present invention. It may also in some instances be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, and dicaprylyl ether.

Particularly advantageous mixtures are those of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate.

Of the hydrocarbons, paraffin oil, squalane and squalene are to be used advantageously for the purposes of the present invention.

The oil phase can advantageously also have a content of cyclic or linear silicone oils, or consist entirely of such oils, although it is preferable to use an additional content of other oil phase components apart from the silicone oil or the silicone oils. Such silicones or silicone oils may be in the form of monomers, which are generally characterized by structural elements, as follows:

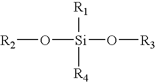

Linear silicones having two or more siloxyl units, which are to be used advantageously according to the invention, are generally characterized by structural elements, as follows:

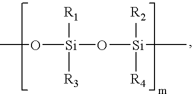

where the silicon atoms can be substituted by identical or different alkyl radicals and/or aryl radicals, which are shown here in general terms by the radicals R₁-R₄ (that is to say the number of different radicals is not necessarily limited to up to 4). m can assume values from 2-200 000.

Cyclic silicones to be used advantageously according to the invention are generally characterized by structural elements, as follows

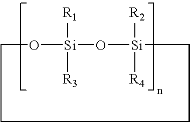

where the silicon atoms can be substituted by identical or different alkyl radicals and/or aryl radicals, which are shown here in general terms by the radicals R₁-R₄ (that is to say the number of different radicals is not necessarily limited to up to 4). n can assume values from 3/2 to 20. Fractions for n take into consideration the fact that uneven numbers of siloxyl groups may be present in the cycle.

Advantageously, cyclomethicone (e.g. decamethylcyclopentasiloxane) is used as the silicone oil to be used according to the invention. However, other silicone oils can also be used advantageously for the purposes of the present invention, for example undecamethylcyclotrisiloxane, polydimethylsiloxane, poly(methylphenyl-siloxane), cetyldimethicone, behenoxydimethicone.

Also advantageous are mixtures of cyclomethicone and isotridecyl isononanoate, and those of cyclomethicone and 2-ethylhexyl isostearate.

It is, however, also advantageous to choose silicone oils of similar constitution to the above-described compounds whose organic side chains are derivatized, for example polyethoxylated and/or polypropoxylated. These include, for example, polysiloxane-polyalkyl-polyether copolymers, such as cetyl-dimethicone copolyol, (cetyl-dimethicone copolyol (and) polyglyceryl-4-isostearate (and) hexyl laurate).

Also particularly advantageous are mixtures of cyclomethicone and isotridecyl isononanoate, and of cyclomethicone and 2-ethylhexyl isostearate.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and, in particular, one or more thickeners which can advantageously be chosen from the group consisting of silicon dioxide and aluminum silicates.

Preparations according to the invention in the form of emulsions or hydrogels advantageously comprise, in particular, one or more hydrocolloids. These hydrocolloids can advantageously be chosen from the group of gums, polysaccharides, cellulose derivatives, phyllosilicates, polyacrylates and/or other polymers.

The gums include saps from plants or trees which harden in the air and form resins, or extracts from aquatic plants. From this group, for the purposes of the present invention, gum arabic, carob flour, tragacanth, karaya, guar gum, pectin, gellan gum, carrageen, agar, algins, chondrus, xanthan gum, for example, can be chosen advantageously.

Also advantageous is the use of derivatized gums, such as, for example, hydroxypropyl guar (Jaguar® HP 8).

The polysaccharides and polysaccharide derivatives include, for example, hyaluronic acid, chitin and chitosan, chondroitin sulfates, starch and starch derivatives.

The cellulose derivatives include, for example, methylcellulose, carboxymethylcellulose, hydroxyethylcellulose, and hydroxypropylmethylcellulose.

The phyllosilicates include naturally occurring and synthetic clay earths, such as, for example, montmorillonite, bentonite, hectorite, laponite, magnesium aluminum silicates such as Veegum®. These can be used as such or in modified form, such as, for example, stearylalkonium hectorites.

In addition, silica gels can also be used advantageously.

The polyacrylates include, for example, Carbopol grades from Goodrich (Carbopol 980, 981, 1382, 5984, 2984, EDT 2001 or Pemulen TR2).

The polymers include, for example, polyacrylamides (Seppigel 305), polyvinyl alcohols, PVP, PVP/VA copolymers, and polyglycols.

Preparations according to the invention in the form of emulsions comprise one or more emulsifiers. These emulsifiers can advantageously be chosen from the group of nonionic, anionic, cationic or amphoteric emulsifiers.

The nonionic emulsifiers include

a) partial fatty acid esters and fatty acid esters of polyhydric alcohols and ethoxylated derivatives thereof (e.g. glyceryl monostearates, sorbitan stearates, glyceryl stearyl citrates, sucrose stearates),

b) ethoxylated fatty alcohols and fatty acids,

c) ethoxylated fatty amines, fatty acid amides, fatty acid alkanolamides, and

d) alkylphenol polyglycol ethers (e.g. Triton X).

The anionic emulsifiers include

e) soaps (e.g. sodium stearate),

f) fatty alcohol sulfates, and

g) mono-, di- and trialkylphosphoric esters and ethoxylates thereof.

The cationic emulsifiers include

a) quaternary ammonium compounds with a long-chain aliphatic radical, e.g. distearyidiammonium chloride.

The amphoteric emulsifiers include

a) alkylaminoalkanecarboxylic acids,

b) betaines, sulfobetaines, and

c) imidazoline derivatives.

In addition, there are naturally occurring emulsifiers, which include beeswax, wool wax, lecithin and sterols.

O/W emulsifiers can be advantageously chosen, for example, from the group of polyethoxylated or polypropoxylated or polyethoxylated and polypropoxylated products, e.g.:

fatty alcohol ethoxylates,

ethoxylated wool wax alcohols,

polyethylene glycol ethers of the general formula R—O—(—CH₂—CH₂—O—)ₙ—R′,

fatty acid ethoxylates of the general formula R—COO—(—CH₂—CH₂—O—)ₙ—H,

etherified fatty acid ethoxylates of the general formula R—COO—(—CH₂—CH₂—O—)ₙ—R′,

esterified fatty acid ethoxylates of the general formula R—COO—(—CH₂—CH₂—O—)ₙ—C(O)—R′,

polyethylene glycol glycerol fatty acid esters,

ethoxylated sorbitan esters,

cholesterol ethoxylates,

ethoxylated triglycerides,

alkyl ether carboxylic acids of the general formula R—O—(—CH₂—CH₂—O—)ₙ—CH₂—COOH and n are a number from 5 to 30,

polyoxyethylene sorbitol fatty acid esters,

alkyl ether sulfates of the general formula R—O—(—CH₂—CH₂—O—)ₙ—SO₃—H,

fatty alcohol propoxylates of the general formula R—O—(—CH₂—CH(CH₃)—O—)ₙ—H,

polypropylene glycol ethers of the general formula R—O—(—CH₂—CH(CH₃)—O—)ₙ—R′,

propoxylated wool wax alcohols,

etherified fatty acid propoxylates R—COO—(—CH₂—CH(CH₃)—O—)ₙ—R′,

esterified fatty acid propoxylates of the general formula R—COO—(—CH₂—CH(CH₃)—O—)ₙ—C(O)—R′,

fatty acid propoxylates of the general formula R—COO—(—CH₂—CH(CH₃)—O—)ₙ—H,

polypropylene glycol glycerol fatty acid esters,

propoxylated sorbitan esters,

cholesterol propoxylates,

propoxylated triglycerides,

alkyl ether carboxylic acids of the general formula R—O—(—CH₂—CH(CH₃)O—)ₙ—CH₂—COOH,

alkyl ether sulfates or the parent acids of these sulfates of the general formula R—O—(—CH₂—CH(CH₃)—O—)ₙ—SO₃—H,

fatty alcohol ethoxylates/propoxylates of the general formula R—O—Xₙ—Yₘ—H,

polypropylene glycol ethers of the general formula R—O—Xₙ—Yₘ—R′,

etherified fatty acid propoxylates of the general formula R—COO—Xₙ—Yₘ—R′,

fatty acid ethoxylates/propoxylates of the general formula R—COO—Xₙ—Yₘ—H.

According to the invention, particularly advantageous polyethoxylated or polypropoxylated or polyethoxylated and polypropoxylated O/W emulsifiers used are those chosen from the group of substances having HLB values of 11-18, very particularly advantageously having HLB values of 14.5-15.5, provided the O/W emulsifiers have saturated radicals R and R′. If the O/W emulsifiers have unsaturated radicals R and/or R′, or if isoalkyl derivatives are present, then the preferred HLB value of such emulsifiers can also be lower or higher.

It is advantageous to choose the fatty alcohol ethoxylates from the group of ethoxylated stearyl alcohols, cetyl alcohols, and cetylstearyl alcohols (cetearyl alcohols). Particular preference is given to polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20), polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20), polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20), polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15), polyethylene glycol(12) lauryl ether (laureth-12), polyethylene glycol(12) isolauryl ether (isolaureth-12), polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol (19) cetylstearyl ether (ceteareth-19), polyethylene glycol(20) cetylstearyl ether (ceteareth-20).

It is also advantageous to choose the fatty acid ethoxylates from the following group polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

The ethoxylated alkyl ether carboxylic acid or salt thereof which can be used is advantageously sodium laureth-11 carboxylate.

Sodium laureth1-4 sulfate can be used advantageously as alkyl ether sulfate.

An advantageous ethoxylated cholesterol derivative which can be used is polyethylene glycol(30) cholesteryl ether. Polyethylene glycol(25) soyasterol has also proven successful.

Ethoxylated triglycerides which can be advantageously used are polyethylene glycol(60) Evening Primrose glycerides.

It is also advantageous to choose the polyethylene glycol glycerol fatty acid esters from the group polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate, polyethylene glycol(20) glyceryl oleate, polyethylene glycol(20) glyceryl isostearate, and polyethylene glycol(18) glyceryl oleate/cocoate.

It is likewise favorable to choose the sorbitan esters from the group polyethylene glycol(20) sorbitan monolaurate, polyethylene glycol(20) sorbitan monostearate, polyethylene glycol(20) sorbitan monoisostearate, polyethylene glycol(20) sorbitan monopalmitate, and polyethylene glycol(20) sorbitan monooleate.

Advantageous W/O emulsifiers which can be used are: fatty alcohols having 8 to 30 carbon atoms, monoglycerol esters of saturated and/or unsaturated, branched and/or unbranched alkanecarboxylic acids having a chain length of from 8 to 24, in particular 12-18, carbon atoms, diglycerol esters of saturated and/or unsaturated, branched and/or unbranched alkanecarboxylic acids having a chain length of from 8 to 24, in particular 12-18, carbon atoms, monoglycerol ethers of saturated and/or unsaturated, branched and/or unbranched alcohols having a chain length of from 8 to 24, in particular 12-18, carbon atoms, diglycerol ethers of saturated and/or unsaturated, branched and/or unbranched alcohols having a chain length of from 8 to 24, in particular 12-18, carbon atoms, propylene glycol esters of saturated and/or unsaturated, branched and/or unbranched alkanecarboxylic acids having a chain length of from 8 to 24, in particular 12-18, carbon atoms, and sorbitan esters of saturated and/or unsaturated, branched and/or unbranched alkanecarboxylic acids having a chain length of from 8 to 24, in particular 12-18, carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprate, glyceryl monocaprylate.

The examples below are intended to illustrate the embodiments of the present inventions. Unless stated to the contrary, the data always refer to % by weight.

Examples 1-10: O/W creams

| | Example number | | | | | | | | | |
|---|---|---|---|---|---|---|---|---|---|---|
| | 1 | 2 | 3 | 4 | 5 | 6 | 7 | 8 | 9 | 10 |
| Glyceryl stearate citrate | 2 | | | | | | | | | |
| Glyceryl stearate, self- | | 5 | | | | | | | | |
| emulsifying | | | | | | | | | | |
| PEG-40 stearate | | | 3 | 2.5 | 0.7 | | | | | |
| PEG-100 stearate | | | | | | 1 | | | | |
| Polyglyceryl-3 | | | 2 | 2.5 | 2 | 3 | | | | |
| diisostearate | | | | | | | | | | |
| Polyglyceryl-3 | | | | | | | 3 | | | |
| methylglucose distearate | | | | | | | | | | |
| Sorbitan stearate | | | | | | | 1 | | | |
| Polyethylene glycol(21) | | | | | | | | 2 | | |
| stearyl ether (steareth-21) | | | | | | | | | | |
| Polyethylene glycol(2) | | | | | | | | 1 | | |
| stearyl ether (steareth-2) | | | | | | | | | | |
| Cetearyl glucoside | | | | | | | | | 2 | |
| Stearic acid | | | | | | | | | | 2.5 |
| Myristyl myristate | 1 | | | | 1 | | | | 1 | |
| Behenyl alcohol | | | | | | | 1 | | | 2 |
| Stearyl alcohol | 2 | 1 | | | | | | | 5 | |
| Cetearyl alcohol | | | | 2 | 2 | | | 2 | | 1 |
| Cetyl alcohol | 1 | | 2 | | | 1 | 1 | | | |
| Hydrogenated coconut | 2 | | | | | 1 | | | | 1 |
| fatty glycerides | | | | | | | | | | |
| (hydrogenated | | | | | | | | | | |
| cocoglycerides) | | | | | | | | | | |
| Shea butter | | 2 | | 1 | | | | | | |
| C12-15 alkyl benzoate | | 3 | 1 | | 3 | | 2 | | 2 | |
| Butylene glycol | 1 | | | 1 | | | | | | 2 |
| dicaprylate/dicaprate | | | | | | | | | | |
| Caprylic/capric | | 1 | 4 | 2 | | | 1 | | | |
| triglycerides | | | | | | | | | | |
| Hydrogenated polydecene | | | | | | | | | 1 | |
| Ethylhexyl coco-fatty acid | 3 | | | | | | | | | 2 |
| ester | | | | | | | | | | |
| Octyldodecanol | | | 1 | | | | | 1 | | |
| Mineral oil | | 1 | | | | | | 3 | | |
| Petroleum jelly | 4 | | | | | 2 | | | | |
| Octamethyltetrasiloxane | | 1 | 3 | 5 | 2 | 1 | | 2 | | |
| (cyclomethicone) | | | | | | | | | | |
| Dimethylpolysiloxane | | | | | 1 | | | | | 1 |
| (dimethicone) | | | | | | | | | | |
| Dicaprylyl ether | 1 | 4 | | | | | | | | |
| Dicaprylyl carbonate | | | | 1 | | | 2 | | | 4 |
| Polydecene | | | | | | 1 | | | 5 | |
| TiO₂ | | | | | 1 | 1 | | | | 1 |
| Ethylhexyl | 3 | 2 | 3 | | 2 | 5 | | | | 3 |
| methoxycinnamate | | | | | | | | | | |
| Ethylhexyltriazone | | 2 | | | | | | | | |
| 4-Methylbenzylidene- | | 2 | | | | | | | | |
| camphor | | | | | | | | | | |
| Butylmethoxydibenzoyl- | | 1 | | | | | | | | |
| methane | | | | | | | | | | |
| Bis-ethylhexyloxyphenol- | 1 | | | | 1 | 2 | | | | |
| methoxyphenyltriazine | | | | | | | | | | |
| Ubiquinone (Q10) | | 0.1 | | | | 0.1 | | | 0.1 | |
| Tocopheryl acetate | 1 | | 0.5 | | | | 0.5 | | | |
| α-Glucosylrutin | | | | | 0.1 | | | | | |
| Biotin | | | | | | 0.1 | | 0.1 | | |
| Aluminum chlorohydrate | 1 | 1 | 3 | 2 | 1 | 1 | 3 | 1 | 0.2 | 0.5 |
| α-Cyclodextrin | 1 | | | | | | | | | |
| β-Cyclodextrin | | 1 | 0.5 | 2 | | | | | 0.5 | |
| γ-Cyclodextrin | | | 2 | | 2 | | | | | |
| Hydroxypropyl-β- | | | | | | 2 | | | | 1 |
| cyclodextrin | | | | | | | | | | |
| Methyl-β-cyclodextrin | | | | | | | | 1 | | |
| Distarch phosphate | 2 | 1 | 4 | 2 | 2 | 4 | 2 | | 2 | 0.5 |
| Trisodium EDTA | 0.2 | | | | | | | 0.2 | 0.1 | |
| Iminodisuccinate | 0.1 | 0.1 | | | 0.3 | 0.3 | 0.5 | 0.1 | | 0.1 |
| Phenoxyethanol | 0.3 | | 0.3 | 0.2 | | 0.8 | 0.4 | 0.5 | | 0.3 |
| p-Hydroxybenzoic alkyl | 0.6 | 0.4 | 0.2 | 0.3 | 0.3 | 0.4 | | | 0.4 | 0.6 |
| ester (paraben) | | | | | | | | | | |
| Hexamidine diisethionate | | | | | 0.04 | 0.05 | | 0.1 | | |
| Diazolidinylurea | 0.25 | | 0.1 | 0.2 | | | 0.2 | | 0.1 | |
| 1,3-Dimethylol-5,5- | | 0.2 | | | | | | | | |
| dimethylhydantoin (DMDM | | | | | | | | | | |
| hydantoin) | | | | | | | | | | |
| Iodopropynyl | 0.1 | | | | 3 | | | 0.25 | | |
| butylcarbamate | | | | | | | | | | |
| Ethanol denatured | 1 | 2 | | | | | 8 | | | 3 |
| 2-Ethylhexyl glycerol ether | | | | | | 3 | | | | |
| (octoxyglycerol) | | | | | | | | | | |
| Xanthan gum | 0.6 | | 0.1 | | | 0.2 | 0.1 | | | |
| Polyacrylic acid | 0.05 | | | | 0.1 | | | 0.1 | | |
| (carbomer) | | | | | | | | | | |
| Polyacrylamide | | 0.2 | | | | | 0.2 | | | |
| Glycerol | 5 | 10 | 5 | 7.5 | 5 | 15 | 5 | 6 | 4 | 2 |
| Butylene glycol | 2 | 1 | | | 2 | | | | 2 | |
| Water-and/or oil-soluble | 0.05 | | | | | | | | | 0.1 |
| dyes | | | | | | | | | | |
| Fillers (distarch | | | | 0.5 | | | | | 5 | 1 |
| phosphate, SiO₂, talc, | | | | | | | | | | |
| aluminum stearate) | | | | | | | | | | |
| Perfume | q. s. | q. s. | q. s. | q. s. | q. s. | q. s. | q. s. | q. s. | q. s. | q. s. |
| Water | ad | ad | ad | ad | ad | ad | ad | ad | ad | ad |
| | 100 | 100 | 100 | 100 | 100 | 100 | 100 | 100 | 100 | 100 |

EXAMPLE 11
W/O cream

| | |
|---|---|
| Polyglyceryl-3 diisostearate | 5.0 |
| Polyglyceryl-2 dipolyhydroxystearate | 2.5 |
| Cetearyl alcohol | 2 |
| Cetyl alcohol | 2 |
| C12-15 alkylbenzoate | 10 |
| Caprylic/capric triglycerides | 5 |
| Octyldodecanol | 7 |
| Octamethyltetrasiloxane | 2 |
| (cyclomethicone) | |
| Lactic acid | 5 |
| Aluminum chlorohydrate | 1 |
| β-Cyclodextrin | 0.5 |
| Distarch phosphate | 1 |
| Phenoxyethanol | 0.1 |
| p-Hydroxybenzoic alkyl ester (paraben) | 0.1 |
| Glycerol | 5 |
| Fillers (distarch phosphate, SiO₂, talc, | 0.2 |
| aluminum stearate) | |
| Perfume | q.s. |
| Water | ad 100 |

EXAMPLE 12
Hydrodispersion/gel cream

| | |
|---|---|
| Hydrodispersion/gel cream | |
| Cetearyl alcohol | 1 |
| Shea butter | 1 |
| Caprylic/capric triglycerides | 2 |
| Octyldodecanol | 1 |
| Octamethyltetrasiloxane | 4 |
| (cyclomethicone) | |
| Dimethylpolysiloxane (dimethicone) | 1 |
| Polydecene | 2 |
| Ethylhexyl methoxycinnamate | 2 |
| Ubiquinone (Q10) | 0.1 |
| Aluminum chlorohydrate | 0.2 |
| β-Cyclodextrin | 0.5 |
| Distarch phosphate | 1 |
| Phenoxyethanol | 0.3 |
| p-Hydroxybenzoic alkyl ester (paraben) | 0.4 |
| Polyacrylic acid (carbomer) | 0.1 |
| Crosslinked alkyl acrylate (alkyl acrylate | 0.2 |
| crosspolymer) | |
| Glycerol | 5 |
| Perfume | q.s. |
| Water | ad 100 |

That which is claimed:

1. A cosmetic or dermatological preparation for removing sebum or for regulating sebum production, comprising:
one or more salts of hard trivalent or tetravalent metal ions; and
one or more unbranched, branched, cyclic or crosslinked oligosaccharides, polysaccharides or derivatives thereof.

2. The preparation as claimed in claim 1, wherein the one or more salts of hard trivalent or tetravalent metal ions include one or more salts selected from the group consisting of aluminum chlorohydrates, aluminum hydroxylactates and acidic aluminum/zirconium salts.

3. The preparation as claimed in claim 1, wherein the oligosaccharides or polysaccharides include oligosaccharides and polysaccharides selected from the group consisting of α-cyclodextrin, β-cyclodextrin, γ-cyclodextrin, phosphated corn starches and mixtures thereof.

4. The preparation as claimed in claim 3, wherein the oligosaccharides or polysaccharides include a mixture of cyclodextrins comprising at least 30% by weight, based on the total weight of the cyclodextrin mixture, of β-cyclodextrin.

5. The preparation as claimed in claim 3, wherein the oligosaccharides or polysaccharides include crosslinked starch phosphate esters.

6. The preparation as claimed in claim 3, wherein the oligosaccharides or polysaccharides include distarch phosphate.

7. The preparation as claimed in claim 1, comprising 0.01-20% by weight, of unbranched, branched, cyclic or crosslinked oligosaccharides, polysaccharides or derivatives thereof.

8. The preparation as claimed in claim 1, comprising 0.1-10% by weight, of said unbranched, branched, cyclic or crosslinked oligosaccharides, polysaccharides or derivatives thereof.

9. The preparation as claimed in claim 1, comprising 0.01-10% by weight, of said salts of trivalent or tetravalent metals.

10. The preparation as claimed in claim 1, comprising 0.05-7% by weight, of said salts of trivalent or tetravalent metals.

11. The preparation as claimed in claim 1, comprising 0.1-5% by weight, of said salts of trivalent or tetravalent metals.

12. A cosmetic or dermatological preparation for removing sebum or for regulating sebum production, comprising:
at least one salt selected from the group consisting of aluminum chlorohydrate, aluminum hydroxylactate, acidic aluminum/zirconium salts, and mixtures thereof; and
at least one cyclodextrin selected from the group consisting of α-cyclodextrin, β-cyclodextrin, γ-cyclodextrin, and mixtures thereof.

13. The preparation as claimed in claim 12, wherein the at least one cyclodextrin includes at least 30% by weight, based on the total weight of the cyclodextrins, of β-cyclodextrin.

14. A cosmetic or dermatological preparation for removing sebum or for regulating sebum production, comprising:
at least one salt selected from the group consisting of aluminum chlorohydrate, aluminum hydroxylactate, acidic aluminum/zirconium salts, and mixtures thereof; and
distarch phosphate.

15. A method for removing sebum or for regulating sebum production, comprising the step of applying a cosmetic or dermatological preparation to the skin or hair, the preparation comprising one or more salts of hard trivalent or tetravalent metal ions and one or more unbranched, branched, cyclic or crosslinked oligosaccharides, polysaccharides or derivatives thereof.

16. The method as claimed in claim 15, wherein the one or more salts of hard trivalent or tetravalent metal ions include one or more salts selected from the group consisting of aluminum chlorohydrates, aluminum hydroxylactates and acidic aluminum/zirconium salts.

17. The method as claimed in claim 15, wherein the oligosaccharides or polysaccharides include oligosaccharides and polysaccharides selected from the group consisting of α-cyclodextrin, β-cyclodextrin, γ-cyclodextrin, phosphated corn starches and mixtures thereof.

18. The method as claimed in claim 17, wherein the oligosaccharides or polysaccharides include a mixture of cyclodextrins comprising at least 30% by weight, based on the total weight of the cyclodextrin mixture, of β-cyclodextrin.

19. The method as claimed in claim 17, wherein the oligosaccharides or polysaccharides include crosslinked starch phosphate esters.

20. The method as claimed in claim 17, wherein the oligosaccharides or polysaccharides include distarch phosphate.

21. The method as claimed in claim 15, comprising 0.01-20% by weight, of unbranched, branched, cyclic or crosslinked oligosaccharides, polysaccharides or derivatives thereof.

22. The method as claimed in claim 15, comprising 0.1-10% by weight, of said unbranched, branched, cyclic or crosslinked oligosaccharides, polysaccharides or derivatives thereof.

23. The method as claimed in claim 15, comprising 0.01-10% by weight, of said salts of trivalent or tetravalent metals.

24. The method as claimed in claim 15, comprising 0.05-7% by weight, of said salts of trivalent or tetravalent metals.

25. The method as claimed in claim 15, comprising 0.1-5% by weight, of said salts of trivalent or tetravalent metals.

26. The method as claimed in claim 15, wherein said preparation is applied to prevent or treat comedones or acne.

27. The method as claimed in claim 15, wherein said preparation is applied to prevent or treat seborrheic phenomena.

28. The method as claimed in claim 15, wherein said preparation is applied to prevent or treat greasy hair.

29. The method as claimed in claim 15, wherein said preparation is applied to prevent or treat dandruff.
